(19) United States
(12) Patent Application Publication
(10) Pub. No.: US 2016/0091476 A1
(43) Pub. Date: Mar. 31, 2016

(54) MOBILE PLUME INTEGRATOR SYSTEM

(71) Applicant: The Regents of The University of California, Oakland, CA (US)

(72) Inventor: Marc L. Fischer, Berkeley, CA (US)

(21) Appl. No.: 14/870,573

(22) Filed: Sep. 30, 2015

Related U.S. Application Data
(60) Provisional application No. 62/057,945, filed on Sep. 30, 2014.

(51) Int. Cl. G01N 33/00

(52) U.S. Cl. CPC G01N 33/0036

(57) ABSTRACT

A system for measuring a gas plume includes a vehicle affixed or attached with a mast comprising a plurality of perforated inlet tubes configured to measure or detect real-time height-resolved sampling of a gas of interest, such as methane, over the height of the mast, and/or a plurality of gas analyzers, wherein the analyzers are configured to provide a continuous readout of the gas of interest from each height interval using the plurality of gas analyzers from each height interval.

### CROSS-REFERENCE TO RELATED APPLICATIONS

This application claims priority to U.S. Provisional Patent Application No. 62/057,945 filed Sep. 30, 2014, titled MOBILE PLUME INTEGRATOR SYSTEM. This prior application is incorporated herein by reference in its entirety and for all purposes.

### STATEMENT OF GOVERNMENTAL SUPPORT

This invention was made with government support under Contract No. DE-AC02-05CH11231 awarded by the United States Department of Energy. The government has certain rights in the invention.

### FIELD OF THE DISCLOSURE

This disclosure relates generally to methods and devices for detecting a gaseous plume.

### BACKGROUND OF THE DISCLOSURE

The presence of methane leaks has been identified using mobile measurements of methane enhancements in air and indirectly estimating leakage rates using estimates of atmospheric mixing using measured meteorological parameters.

### SUMMARY OF THE DISCLOSURE

The present disclosure provides for a system for measuring a gas plume comprising a vehicle affixed or attached with a mast comprising a plurality of perforated inlet tubes, such as a mast system using multiple perforated inlet tubes, configured to measure or detect real-time height-resolved sampling of a gas of interest, such as methane, over the height of the mast, and/or a plurality of gas analyzers, wherein the analyzers are configured to provide a continuous readout of the gas of interest from each height interval using the plurality of gas analyzer from each height interval. The gas of interest is any gas that can be detected by a gas analyzer. In some embodiments, the gas of interest is methane or CH₄.

The present disclosure provides for a method of measuring a gas plume comprising: (a) providing the system of the present disclosure, (b) moving or driving the vehicle, and (c) measuring or detecting a gas of interest with the gas analyzers, whereby the continuous readout of the gas of interest from each height interval using the plurality of gas analyzer from each height interval is obtained.

These and other objects, advantages, and features of the disclosure will become apparent to those persons skilled in the art upon reading the details of the disclosure as more fully described below.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1 shows a top schematic view of a MPI vehicle in accordance with one embodiment moving through the path of a methane (CH₄) plume emitted by a localized source of CH₄ leakage that is advected across the vehicle motion by wind motion of the ambient air.

FIG. 2 shows a side schematic view of a MPI vehicle in accordance with one embodiment showing sample tubes sampling air over three ranges of height in the vertical for analysis by gas analyzers, represented as shaded rectangles on the left, in the vehicle, while measured winds perpendicular to the vehicle motion are shown as arrows. A graph of measured CH₄ enhancements is shown on the right for each height along the vehicle motion.

FIG. 3 shows an image of a CH₄ plume measured by the MPI system, showing that the CH₄ plume is captured in the horizontal and vertical by the MPI system (note different horizontal and vertical length scales).

### DETAILED DESCRIPTION

It is to be understood that this disclosure is not limited to particular embodiments described, as such may, of course, vary. It is also to be understood that the terminology used herein is for the purpose of describing particular embodiments only, and is not intended to be limiting, since the scope of a presently claimed invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are now described. All publications mentioned herein are incorporated herein by reference to disclose and describe the methods and/or materials in connection with which the publications are cited.

The term “about” refers to a value including 10% more than the stated value and 10% less than the stated value.

In some embodiments, the system comprises a Mobile Plume Integrator (MPI) system. The MPI system comprises vehicle-mounted detection and analysis equipment and provides rapid quantitative measurement of gases, for example from potential sources distributed over large distances. One particular application of the system is with regard to methane emissions from numerous localized sources that are spatially distributed over large distances, and this disclosure is made primarily in this context. However, one skilled in the art will recognize that the systems and methods could be readily adapted for detection of other gases.

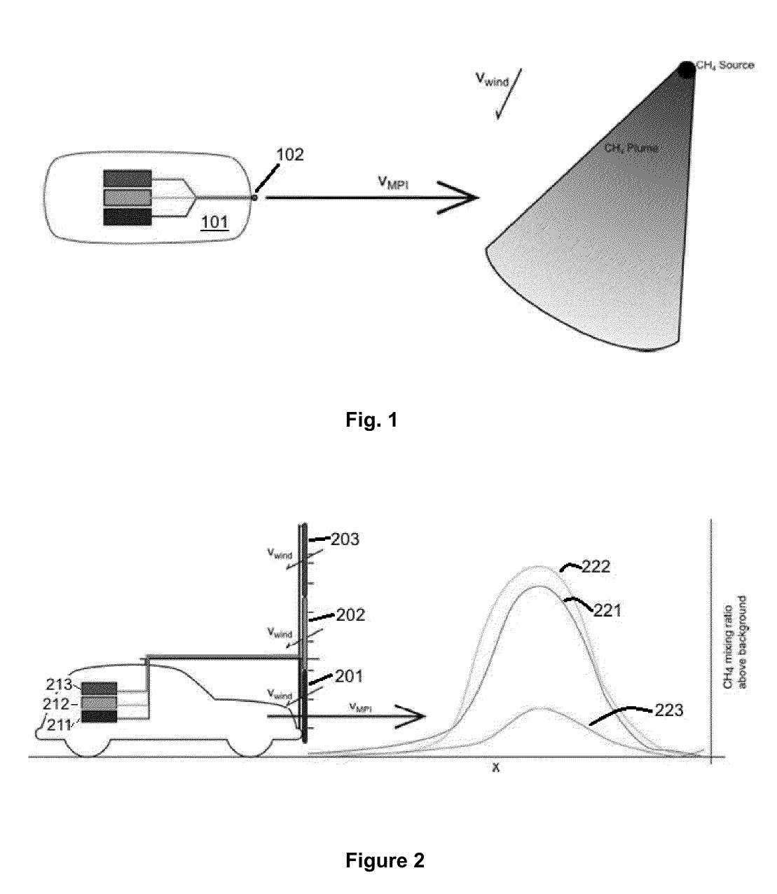

The system and method disclosed facilitates quantitative surveys of methane leakage from natural gas infrastructure, a critical need for both public safety and climate change mitigation. The MPI features continuous real-time measurements of the vertical distribution of methane (CH₄) and wind substantially perpendicular to the motion of a vehicle-borne detection equipment, both of which are used to compute the wind-borne advective flux of a gas, such as CH₄. FIG. 1 shows a top schematic view of a MPI vehicle 101 in accordance with one embodiment moving through the path of a methane (CH₄) plume emitted by a localized source of CH₄ leakage that is advected across the vehicle motion by wind motion of the ambient air. Gas detection apparatus can be mounted on a mast 102 mounted to the vehicle 101.

For the purpose of measuring total emissions from plumes in open air, the system can measure both the vertical and horizontal extents of the plumes. FIG. 2 shows a side schematic view of the MPI vehicle 101 showing sampling apparatus mounted on the vertical mast 102 for sampling air over three ranges of height in the vertical, 201, 202, 203, for analysis by corresponding gas analyzers in the vehicle 211, 212, 213, respectively, represented as shaded rectangles on the left, while measured winds perpendicular to the vehicle motion are shown as arrows. A sonic anemometer can be deployed on the mast to record wind velocity and a GPS unit can measure the vehicles location and velocity. A graph of measured CH₄ enhancements is shown on the right for each height along the vehicle motion, with corresponding plot lines 221, 221, 223, respectively. To provide rapid vertical and horizontal sampling, the MPI can be configured to continuously sample air from multiple heights on an extensible mast which is moved through the air by the vehicle, and then measure the multiple time-varying gas concentrations in real-time together with the vehicle and air motions.

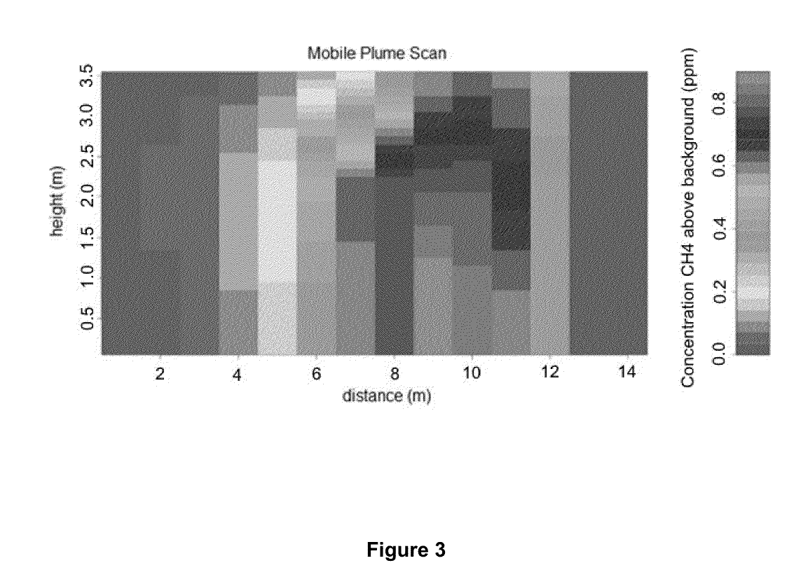

In a particular example, three separate perforated sample tubes collected air over vertical extents of 0.25-1.5 m, 1.5-2.75 m, and 2.75-4 m with inlet holes distributed approximately every 15 cm along each sample tube. In each case, the sample air streams from each tube were measured at approximately 1 Hz using independent closed-cell gas analyzers located inside the MPI vehicle. In addition to the gas concentration measurements, wind speeds were measured using a 3-D sonic anemometer deployed at 2 m in height about the ground, and the location and velocity of the vehicle were measured using a GPS unit. The ambient wind velocity was determined as the difference of the measured wind and the vehicle's motion. All data was recorded and merged into time synchronized data records at a sample rate of about 1 Hz, sufficient to capture the spatial variation of the gas plume and position and wind data. FIG. 3 shows a plot of the example plume indicating the 2-D distribution of CH₄ measured by the MPI system.

Total methane emission in each plume was calculated as the area-integrated flux of CH₄ crossing the plane defined by the motion of the mast as E=∫∫₀^{h} (C−Co)v_{wind} dxdz, where C is measured CH₄ signal, Co is the CH₄ background, v_{wind} is the component of the wind perpendicular to the direction of the vehicle motion, and the area integral is performed over the horizontal and vertical extent of the CH₄ plume.

In some embodiments, the system of the present disclosure can be used for the mitigation of natural gas emissions from production, processing, and/or distribution systems. In some embodiments, the system of the present disclosure can survey and/or quantify the volumetric emission rate of methane, or any other detectable gas, from individual localized components to within about 1 to about 10 meters in the natural gas system, or any other gas system. In some embodiments, the system of the present disclosure can make measurements from roadways on distances of scales about or at least ten kilometers, or 20, 30, 40, or 50 km, over time scales of about or at least one hour, or 2, 3, 4, 5, or 6 hours.

The Mobile Plume Integrator (MPI) system makes rapid direct measurements of the vertical and horizontal distribution of atmospheric enhancements of gas or aerosol species and wind velocity in a plane defined by the motion of a moving vehicle. The total emissions from the leakage source is then quantified as the area-integrated advective flux of each species moving perpendicular to the measurement plane.

One feature of system embodiment in accordance with this disclosure is that it utilizes multiple gas analyzers to simultaneously measure the atmospheric enhancements at multiple levels with high time resolution in a continuous fashion. Embodiments can also utilize a mast that is configured such that the measurement plane defined by the mast can be either relatively short (e.g., about 4 m) for use in most urban roadways, and taller (e.g., about 10 m) for use in unobstructed areas. In some embodiments, the height of the mast can extensible, such as from about 4 m to about 10 m. This taller or extensible embodiment allows the MPI system to capture plumes that have greater vertical extent.

In some embodiments, the MPI system is useful for rapid and accurate measurement of gas leakage from localized leaks. This is valuable for assessing the magnitude of total leakage from facilities and urban environments.

While the present disclosure has been described with reference to the specific embodiments, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the claims appended hereto.

What we claim is:

1. A system for measuring a gas plume comprising a vehicle affixed or attached with a mast comprising a plurality of perforated inlet tubes configured to measure or detect real-time height-resolved sampling of a gas of interest over the height of the mast, and/or a plurality of gas analyzers, wherein the analyzers are configured to provide a continuous readout of the gas of interest from each height interval using the plurality of gas analyzer from each height interval.

2. The system of claim 1, wherein the gas is methane.

3. The system of claim 1, wherein mast has a height of up to about 4 m above ground level.

4. The system of claim 1, wherein mast has a height of up to about 10 m above ground level.

5. The system of claim 1, wherein mast is extensible from about 4 m to about 10 m above ground level.

6. A method of measuring a gas plume comprising:
(a) providing the system of claim 1,
(b) moving or driving the vehicle, and
(c) measuring or detecting a gas of interest with the gas analyzers;
whereby the continuous readout of the gas of interest from each height interval using the plurality of gas analyzers from each height interval is obtained.

7. The method of claim 3, wherein the gas is methane.

8. The method of claim 1, wherein mast has a height of up to about 4 m above ground level.

9. The method of claim 1, wherein mast has a height of up to about 10 m above ground level.

10. The method of claim 1, wherein mast is extensible from about 4 m to about 10 m above ground level.
